(19) United States
(12) Patent Application Publication
(10) Pub. No.: US 2005/0059967 A1
(43) Pub. Date: Mar. 17, 2005

(54) Electrosurgical device

(76) Inventors: Earl E. Breazeale JR., Maryville, TN (US); Warren S. Bennett, Strawberry Plains, TN (US); Eric A. Ward, Knoxville, TN (US)

Correspondence Address: LUEDEKA, NEELY & GRAHAM, P.C., P O BOX 1871, KNOXVILLE, TN, 37901, US

(21) Appl. No.: 10/752,997

(22) Filed: Jan. 7, 2004

Related U.S. Application Data
(63) Continuation-in-part of application No. 29/189,853, filed on Sep. 11, 2003, now Pat. No. D501047.

(51) Int. Cl. A61B 18/14

(52) U.S. Cl. 606/45; 606/49

(57) ABSTRACT

An electrosurgical device including a switch assembly having electronic circuitry positioned in a first plane, and a blade in electrical communication with the circuitry and fixedly positioned in a second plane spaced vertically apart from the first plane and substantially parallel thereto.

### CROSS-REFERENCE TO RELATED APPLICATION

This is a continuation-in-part of U.S. Design Patent Application Ser. No. 29/189,853 entitled CAUTERY DEVICE and filed Sep. 11, 2003.

### FIELD OF THE INVENTION

This invention relates generally to electrosurgical devices. More particularly, the invention relates to electrosurgical cautery devices.

### BACKGROUND AND SUMMARY OF THE INVENTION

Handheld electrosurgical cautery devices desire improvement in their configuration to render them more readily controlled by a user. For example, such devices may be difficult to grip in a manner which enables fine positioning during use. It is also often difficult for a user to manipulate the electrical switch of the device in a desirable manner while positioning the device. With regard to the foregoing, the present invention is directed to an electrosurgical device.

In a preferred embodiment, the device includes a switch assembly having electronic circuitry positioned in a first plane, and a blade in electrical communication with the circuitry and fixedly positioned in a second plane spaced vertically apart from the first plane and substantially parallel thereto.

In another aspect, the invention relates to a housing for an electrosurgical cautery device. The housing preferably includes an electrically insulative elongate body having a forward tip end an aperture defined therethough for receiving a mode selection associated with an electrical circuit to be housed by the housing. The distance between a forward most portion of the aperture and the tip end of the housing is preferably less than about 0.6 inches, most preferably between about 0.4 and 0.5 inches.

In yet another aspect, the invention relates to a coupler for coupling an electrosurgical blade to an electrical circuit. In a preferred embodiment, the coupler includes a conductive material having a first end configured to receive a portion of the blade and a second end configured to be engageable with a portion of the circuit. The second end also includes a substantially J-shaped portion that connects to an intermediate segment that bridges between the first and second ends.

### BRIEF DESCRIPTION OF THE DRAWINGS

Further features of preferred embodiments of the invention will become apparent by reference to the detailed description of preferred embodiments when considered in conjunction with the figures, which are not to scale, wherein like reference numbers, indicate like elements through the several views, and wherein,

FIG. 1 is a perspective view of an electrosurgical device according to a preferred embodiment of the invention.

FIG. 2 is a top plan view of the device of FIG. 1.

FIG. 3 is a bottom plan view of the device of FIG. 1.

FIG. 4 is a right side view of the device of FIG. 1.

FIG. 5 is a partial exploded perspective view of the device of FIG. 1.

FIG. 6 is an exploded perspective view of the cautery device of FIG. 1.

FIG. 7 is perspective view of a coupler for use with the device of FIG. 1.

FIG. 8 is a detailed view showing connection of the coupler of FIG. 7 to a circuit board of the device of FIG. 1.

FIGS. 9A-9C show details of a lower body portion of a housing of the device of FIG. 1.

FIGS. 10A-10C show details of a upper body portion of a housing of the device of FIG. 1.

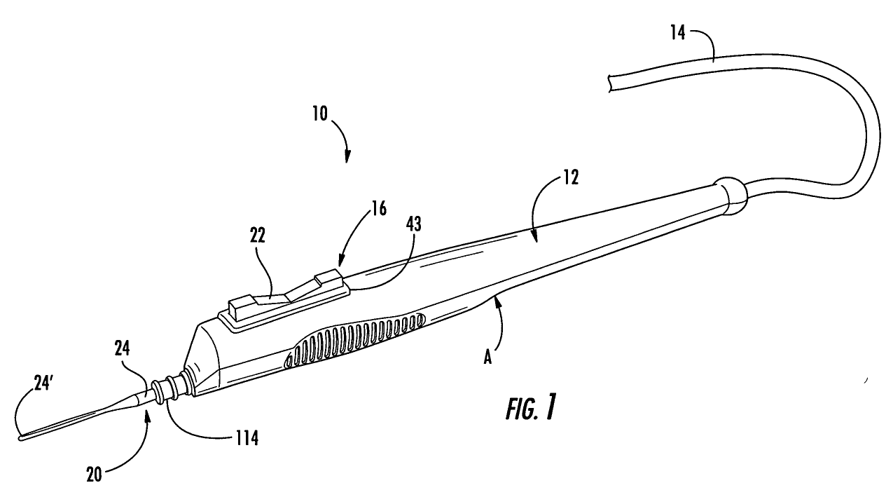

### DETAILED DESCRIPTION

With reference to the drawings, the invention relates to an electrosurgical device 10 having a housing 12, an electrical cable 14, a switch assembly 16, a blade-holder or coupler 18, and a blade 20. A significant aspect of the invention relates to the relative short displacement between a mode selection button 22 associated with the switch assembly 16, and an operative portion 24 of the blade 20.

The housing 12 is preferably of two-piece plastic construction and includes first and second mating elongate body portions 30 and 32. The body portions 30 and 32 may be snap-fit together, mated by sonic welding, and by other conventional ways of joining mating plastic parts. The portions 30 and 32 are configured for housing the electrical switch 16 and providing access for the cable 14, the coupler 18 and a portion of the blade 20.

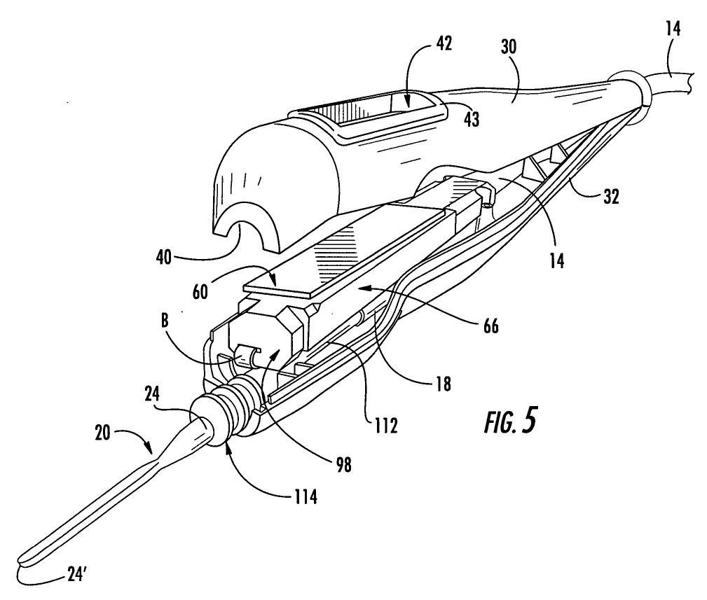

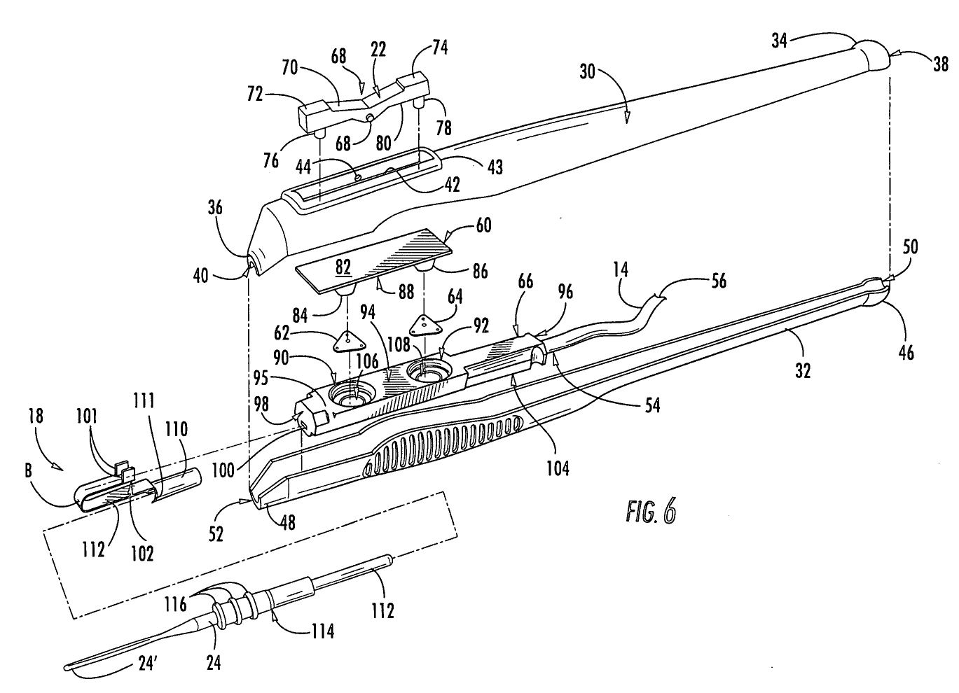

With additional reference to FIG. 6, the first body portion 30 includes a rear end 34 and an opposite tip end 36. The rear end 34 defines a cable aperture portion 38 and the tip end 36 defines a blade aperture portion 40. A switch aperture 42 extends through the body portion 30 adjacent the tip end 36. A raised window or frame 43 preferably surrounds the aperture 42 for aesthetics and to add additional material to help retain the button 22 laterally.

The body portions 30 and 32 may be shaped and otherwise configured for aesthetics and to facilitate gripping thereof and to provide an aesthetically pleasing appearance. For example, the housing 12 preferably includes various contours and topographies, such as the topographies 41, with the dimension of the housing 12 being generally tapered and increasing in dimension from the rear end 34 to the tip end 36, and most preferably having a substantially abrupt increase in dimension, at point A. For example, the thickness or height of the housing increases by about fifty percent, increasing from about 0.5 inches to about 0.75 inches. Also, an underside portion 45 of the housing 12 immediately rearward of the abrupt increase indicated by point A is preferably substantially planar.

The switch aperture 42 is configured according to the button 22 and preferably includes holes or slots 44 on opposite sides thereof for mounting of a portion of the switch assembly 16, as explained below. The aperture 42 may be of various configurations, e.g., rectangular, round, etc., depending upon the switch assembly used. For example, in one alternative, the button 22 may be a pair of buttons and the aperture 42 a pair of apertures.

The second body portion 32 is configured to mate with the first body portion 30 and includes a corresponding rear end 46 and an opposite tip end 48. The rear end 46 defines a cable aperture portion 50 and the tip end 48 defines a blade aperture portion 52.

The electrical cable 14 is preferably a conventional electrical cable of the type used for electro-surgical cautery devices and includes an end 54 placeable in electrical communication with the switch assembly 16 and an opposite end 56 connectable to an electrical power device for supplying electrical power to the device 10. The electrical power device may preferably be an electrical generator which provides a high frequency signal which is transmitted to the blade 20 via the cable 14 and a circuit associated with the switch assembly 16. For cutting purposes, the generator may supply a continuous signal. For coagulation, the generator may supply a pulsed signal. The switch assembly 16 enables selection of a cutting signal, a coagulation signal, or no signal corresponding to an “off” condition.

The switch assembly 16 preferably includes the selection button 22 and a membrane 60, a pair of domes 62 and 64, and a overmolded circuit 66. In operation, the button 22 is activated to select cutting or coagulation signals via the circuit 66. In the absence of a selection, no power is supplied. The domes 62 and 64 are made of a flexible electrically conductive sheet material, such as thin metal, and flex when depressed by the projections 84 and 86 to make contact with electrical contacts of the circuit 66 to complete an electrical circuit. Alternatively, the projections 84 and 86 may be configured to be integral with the button 22.

In this regard, while the button 22 is shown as a rocker type button of molded plastic construction, it will be understood that it and its associated structure could be of a variety of configurations so as to be suitable for manipulation by a user to serve to enable the device 10 to be turned of and off, i.e., such as push-buttons or other common switch components.

Returning to FIG. 6, the button 22 includes a pair of pegs 68 located on opposite sides thereof substantially proximate the midpoint of the length of the button 22 for being received within the slots 44 of the aperture 42 to support the button 22 relative to the aperture 42 and to enable the button 22 to be rocked back and forth by a user.

The button 22 includes a user contact surface 70 having plateaus 72 and 74 at opposite ends thereof for being contacted by a user to rock the button 22 between first and second depressed or activated positions, such as between a first position for selecting electrical power suitable for cutting purposes and a second position for selecting electrical power suitable for coagulation purposes. The user may apply pressure to maintain the button in the desired position and, upon release, the button will return to a neutral position wherein no power is supplied.

Projections 76 and 78 project from an opposite surface 80 at locations below the plateaus 72 and 74. The projections 76 and 78 are configured to urge against respective portions of a surface 82 of the membrane 60 when the button 22 is manipulated by depression of the plateau 72 or the plateau 74.

The membrane 60 is made of a flexible, electrically non-conductive material, such as rubber. The membrane 60 is preferably of one-piece construction and includes substantially rigid projections 84 and 86 also preferably made of rubber and extending from an opposite surface 88 thereof at locations generally aligned with the projections 76 and 78. The projections 84 and 86 are configured to contact the domes 62 and 64, respectively, which are received within apertures 90 and 92, respectively, defined through a surface 94 of the overmolded circuit 66.

The overmolded circuit 66 provides an electrical circuit board 66a (FIG. 8) substantially encased in an electrically nonconductive housing 95, preferably as by overmolding the circuit board with a non-conductive plastic material. The overmolded circuit 66 includes an input end 96 into which the end 54 of the cable 14 extends for electrical communication with the encased circuit board 66a and an opposite output end 98 having an aperture 100 through which the coupler 18 extends. In this regard, wit will be understood that the aperture 100 is formed during molding as the molding process encases the circuit board 66a and a connecting portion 102 of the coupler 18 which is electrically connected to the board 66a.

With reference to FIGS. 7 and 8, the connecting end 102 of the coupler 18 preferably includes ears 101 which fit into corresponding apertures 103 of the board 66a and may be bent down to provide a mechanical connection. The end 102 may also be welded or soldered in place to enhance electrical communication.

Returning to FIG. 6, a lower surface 104 of the overmolded circuit 66 is located opposite the surface 94 and the coupler 18 is situated adjacent the surface 104 in the assembled device 10. The apertures 90 and 92 provide access to electrical contacts 106 and 108 of the overmolded circuit board. Activation of the contact 106 preferably enables application of a cutting signal and activation of the contact 108 preferably enables application of a coagulation signal. The general construction of circuit boards suitable for cautery devices and the encasement thereof, are well known in the art.

The domes 62 and 64 flex when depressed by the projections 84 and 86 (as urged by the projections 76 and 78) and flex to make contact with electrical contacts 106 and 108, respectively, of the circuit 66 to complete an electrical circuit for supplying desired power amounts to the blade 20. For example, a user may hold down or depress the plateau 72 to urge the projection 86 against the dome 62 and thereby flex the dome 62 downwardly to make contact with the contact 106 of the circuit 66 and complete an electrical circuit configured to provide cutting power to the blade 20. Likewise, depression of the plateau 74 preferably supplies coagulation power to the blade 20. Absent depression of one of the plateaus 72 or 74, the circuit is open and no power is supplied to the blade 20.

The coupler 18 is preferably of one-piece construction and made of a relatively rigid and electrically conductive material such as copper. The coupler 18 includes a sleeve 110 from which extends a strip 112. Circumferential edge 111 of the sleeve is preferably flared outwardly.

FIG. 7 shows the coupler 18 in a pre-assembly configuration. The coupler 18 preferably has the following length dimensions:

| Dimension | Distance (inches) |
|---|---|
| D1 | 0.34 |
| D2 | 0.70 |
| D3 | 0.08 |

After installation of the coupler onto the board 66a, as by connecting the ears 101 to the apertures 103 and applying a solder to enhance electrical communication, a portion of the strip 112 of the coupler 18 is preferably shaped as by bending to provide bend B to form the coupler 18 into a generally J-shape, wherein the strip 112 and the sleeve 100 of the coupler 18 are positioned below and closely adjacent the surface 104 of the overmolded circuit 66 (FIG. 5).

The blade 20 is a small surgical electrode with the operative end 24 preferably configured to provide a thin knife-like tip 24′which may be applied to a patient (who is generally grounded) during a surgical procedure. The blade 20 is generally replaceable and may be available in a variety of lengths and configurations. The small contact area of the portion of the tip 24′, when supplied with appropriate electrical current, enables cutting or cauterizing functions. The operative end 24 begins immediately adjacent the fitting 114 and extends to the end of the tip 24′. Thus, the distances described herein which reference the “operative end” of the blade will be understood to relate to the shortest distance to the operative end, i.e., to the beginning of the operative end and to be independent of the actual length of the operative end.

The blade 20 also includes a non-operative connecting end 112 and an electrically insulative collet or fitting 114 frictionally located on the blade 20, as by molding, between the non-operative end 112 and the operative end 24. The end 112 is configure, preferably as a cylinder, to be received within the sleeve 110 of the coupler 18 and the fitting 114 is made of an electrically insulating material such as a plastic material. The fitting 114 is configured to be fittingly received by the blade aperture portions 40 and 52 when the body portions 30 and 32 of the housing 12. In this regard, the collet 114 preferably includes a series of rings 116 which facilitate fixed positioning of the blade 20 relative to the housing 12.

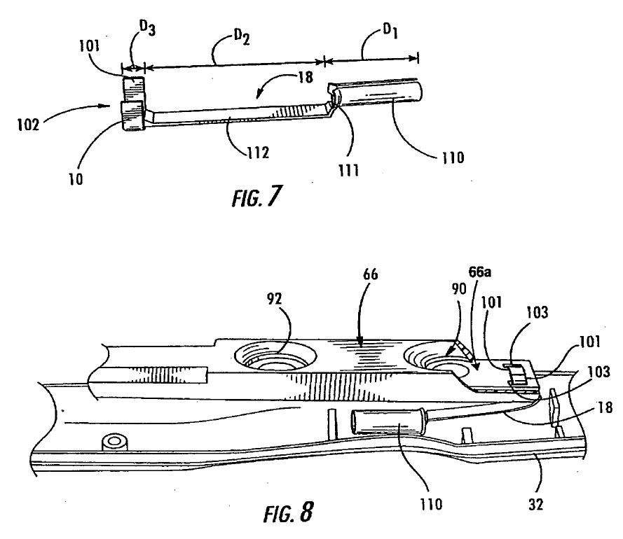

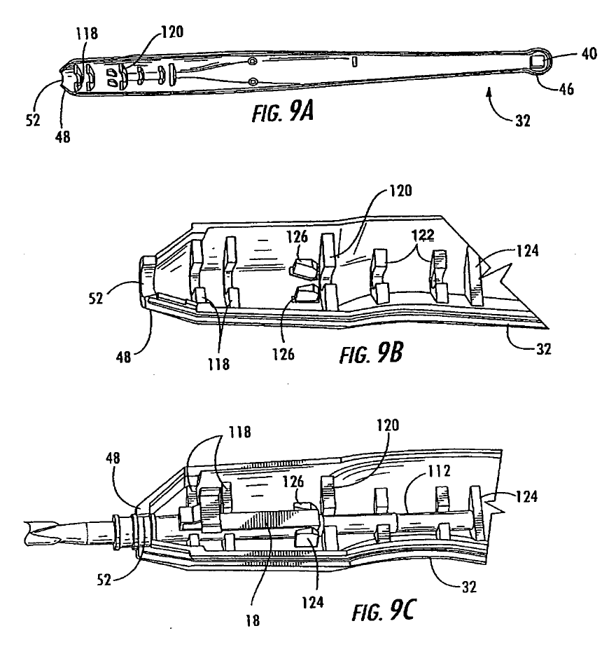

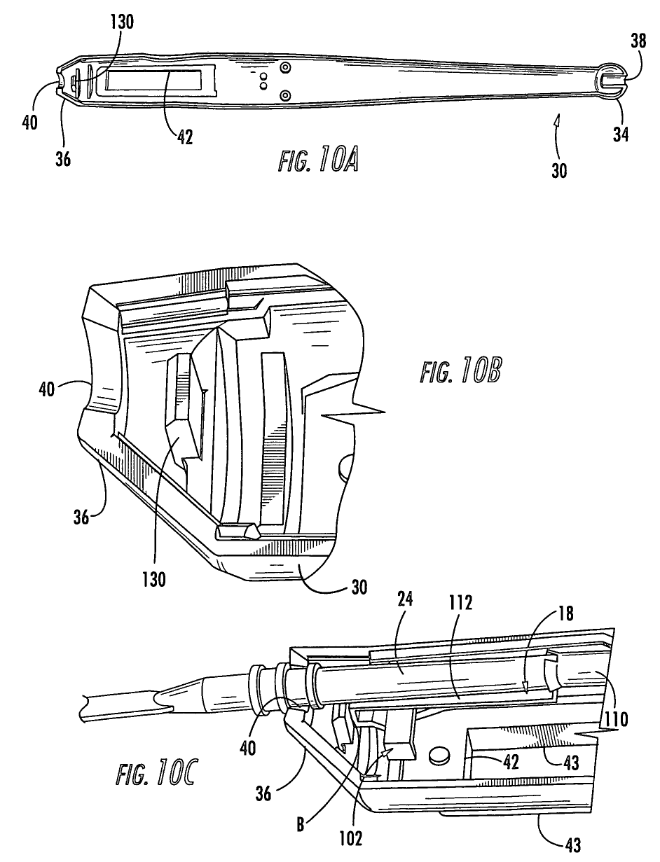

Turning now to FIGS. 9A-9C and 10A-10C, internal details of the body portions 30 and 32 of the housing 12 are configured to inhibit movement of the circuit, the coupler, and the blade. For example, the body portion 32 (FIGS. 9A-9C) includes channeled ribs 118 for snugly receiving the fitting 114, channeled rib 120 for receiving the coupler 18, and channel ribs 122 for receiving the end 112. A stop or wall 124 is located behind the rearmost rib 120 to provide a barrier to rearward movement of the end 112. In addition, it is preferred to include a pair of fingers 126 adjacent the forward edge of the rib 120 and spaced slightly forward thereof.

The fingers 126 are configured, preferably as by tapering toward the rib 120, to enable passage of the flared circumferential edge 111 of the sleeve 110 during installation of the coupler 18 onto the body portion 32. However, once the coupler 18 is installed, the flared edge 111 is located in the space between the fingers 126 and the rib 120 and is of a larger dimension than the smallest passage dimension of the fingers. In this regard, it is noted that the flared edge 111 is preferably initially slightly greater in dimension than the smallest passage dimension of the fingers, yet expands to an even greater dimension when the blade 24 is inserted. However, during installation of the coupler 18, the edge 111 is able to flex inwardly an amount sufficient to enable passage between the fingers 126.

The body portion 30 (FIGS. 10A-10C) preferably includes an internal wall 130 adjacent the forward tip end 36 and positioned between the aperture portion 40 and the aperture 42 so as to provide a barrier against forward movement (movement toward the tip end 36) of the coupler 18 in the assembled device 10. In this regard, the wall 130 is located and configured to abut the bend B of the installed coupler 18.

A significant aspect of the invention relates to the location of the aperture 42 relative to the tip 36 of the housing 12, and the configuration of the coupler 18 which cooperate to enable the relatively short displacement, as compared to prior devices, between the on/off button 22 and the operative portion 24 of the blade 20. The device 10 enables the blade 20 to be located below the circuit 66 in the assembled device 10, advantageously providing a configuration wherein the distances between the components of the device activated to provide power to the blade 20, such as the plateaus 70 and 72, and the operative end 24 of the blade 20 is reduced as compared to conventional cautery devices.

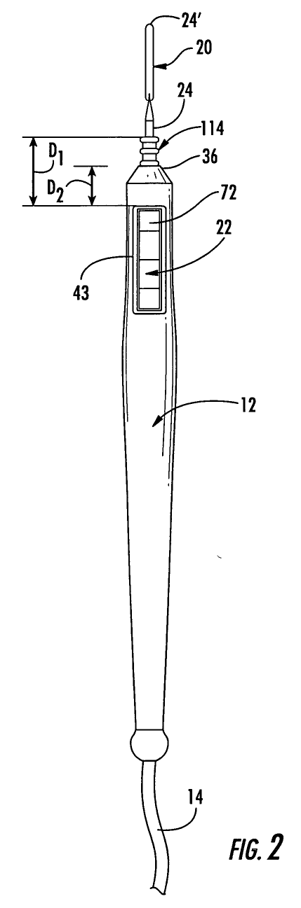

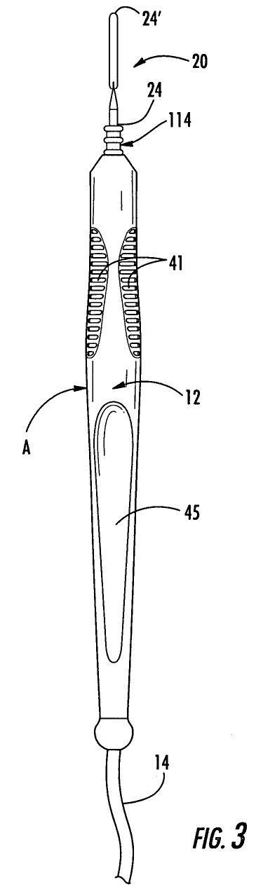

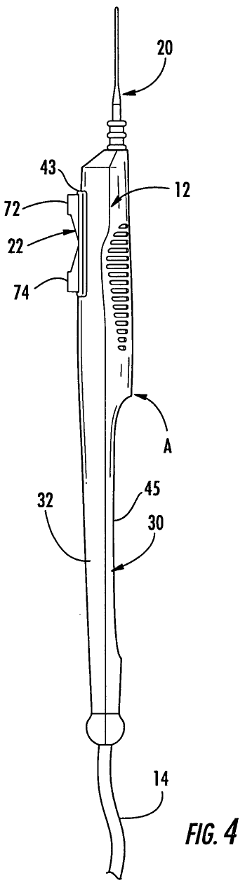

For example, with reference to FIG. 2, the distance D1 between a portion of the button 22 and the operative end 24 of the blade 20 is preferably less than about 0.9 inches and is most preferably from about 0.7 to about 0.85 inches. The distance D2 defined between a portion of the button 22 and tip end 36 of the housing 12 is preferably less than about 0.6 inches and is most preferably from about 0.4 to about 0.5 inches.

The foregoing description of certain exemplary embodiments of the present invention has been provided for purposes of illustration only, and it is understood that numerous modifications or alterations may be made in and to the illustrated embodiments without departing from the spirit and scope of the invention as defined in the following claims.

1. An electrosurgical device, comprising a switch assembly having electronic circuitry positioned in a first plane, and a blade electrically connected to the circuitry by an electrically conductive coupler extending from an end of the circuitry, the coupler having a sleeve configured to receive a portion of the blade, a strip having a first end extending from the sleeve and a substantially J-shaped opposite second end directly connected to the circuitry, wherein the sleeve is fixedly positioned in a second plane spaced vertically apart from the first plane and substantially parallel thereto.

2. The device of claim 1, wherein the second plane is below the first plane.

3. (cancelled).

4. (cancelled).

5. The device of claim 1, further comprising a housing having a tip end and a mode selection button associated with the electrical circuit and extending through an aperture of the housing, with the blade including an operative end extending outwardly of the tip end of the housing, wherein the shortest distance between a portion of the button and the operative end of the blade is no more than about 0.9 inches.

6. The device of claim 1, further comprising a housing having a tip end and a mode selection button associated with the electrical circuit and extending through an aperture of the housing, wherein the shortest distance between a portion of the button and the tip end of the housing is no more than about 0.6 inches.

7. A housing for an electrosurgical cautery device, the housing comprising an electrically insulative elongate body having a forward tip end and an aperture defined thorough the body for receiving a mode selection button associated with an electrical circuit to be housed by the housing, wherein the shortest distance between a forward most portion of the aperture and the tip end of the housing is no more than about 0.6 inches, and wherein the aperture includes a raised window substantially surrounding the aperture to help retain the button laterally.

8. The housing of claim 7, wherein the housing includes a rear end, with the housing having a generally tapered exterior dimension which increases from the rear end to the tip end.

9. The housing of claim 8, wherein the housing includes a substantially abrupt increase in dimension at a location intermediate the ends and an underside portion adjacent the location of the abrupt increase in dimension which is substantially planar.

10. A coupler for coupling an electrosurgical blade to an electrical circuit, the coupler comprising a conductive material having a first end configured to receive a portion of the blade, a second end configured to be engageable with a portion of the circuit and including a substantially J-shaped portion which connects to an intermediate segment that bridges between the first and second ends.

11. The coupler of claim 10, wherein the first end comprises a sleeve and the intermediate segment comprises a substantially linear strip.
